(19) United States
(12) Patent Application Publication
(10) Pub. No.: US 2018/0214837 A1
(43) Pub. Date: Aug. 2, 2018

(54) TWO-STAGE REACTOR FOR EXOTHERMAL AND REVERSIBLE REACTIONS AND METHODS THEREOF

(71) Applicant: PRIMUS GREEN ENERGY INC., Hillsborough, NJ (US)

(72) Inventors: Eli GAL, Sunnyvale, CA (US); Robert M. KOROS, Westfield, NJ (US); Benjamin MOSKOWITZ, Monmouth Junction, NJ (US)

(21) Appl. No.: 15/748,487

(22) PCT Filed: Jul. 29, 2016
(86) PCT No.: PCT/US16/44720

§ 371 Date: Jan. 29, 2018

Related U.S. Application Data
(60) Provisional application No. 62/198,348, filed on Jul. 29, 2015.

(51) Int. Cl. B01J 8/06; F28D 7/16; F28D 7/00; C07C 29/152

(52) U.S. Cl. CPC B01J 8/067; F28D 7/16; F28D 2021/0022; C07C 29/152; F28D 7/0083

(57) ABSTRACT

The present invention relates a two-stage reactor for exothermal, reversible reactions. In particular, the reactor contains a first semi-isothermal stage followed by a second cooling stage. The reactor allows for high conversion of products in an exothermal, reversible reaction.

### CROSS-REFERENCE TO RELATED APPLICATION

This application claims the priority of U.S. Provisional Patent Application No. 62/198,348, filed Jul. 29, 2015, which is incorporated herein by reference.

### FIELD OF THE INVENTION

The present invention relates to a two-stage reactor for exothermal, reversible reactions. In particular, the reactor contains a first semi-isothermal stage followed by a second cooling stage. The reactor allows for high conversion of products in exothermal, reversible reactions.

### BACKGROUND

Isothermal or pseudo-isothermal chemical reactors are reactors fitted with an internal heat exchanger, usually embedded in a catalytic rack, to keep the temperature of the chemical reaction in an optimum range. A common example is the synthesis of methanol, where the heat exchanger removes the heat of the exothermic reaction with a suitable cooling fluid, e.g. by converting boiling water into steam.

In a first known arrangement, a tubular reactor is basically a shell containing a fixed tube bundle, and a catalyst accommodated inside the tubes. The shell also contains boiling water at a single constant pressure and temperature.

For reversible, exothermic reactions (endothermic in the reverse reaction), the conversion of reactants to products is limited by the rate of the forward reaction which is faster at higher temperature, and by equilibrium which favors the reverse reaction at high temperature. Thus, at high temperature, reaction rate and equilibrium work against each other resulting in poor product conversion.

Therefore, there remains a need for a chemical reactor that can be operated to take advantage of high reaction rate at high temperature, and to improve equilibrium conditions when significant products are in the reaction mixture.

### SUMMARY OF THE INVENTION

An aspect of the present invention provides a high efficiency two-stage, single reactor for exothermal and reversible reactions, such as the production of methanol from synthesis gas (syngas). The reactor is a shell and tube vessel. Process gas or liquid flows in the tube and react to produce products with or without the help of a catalyst. The exothermic reaction in the tube is cooled by transferring the heat to colder fluid in the shell. The cooling fluid in the shell is heated by the heat transfer from the heat generated by the reaction in the tubes to produce vapor at a constant temperature and pressure. The reactor is separated into two different zones separated by a device that prevents back mixing of the fluid in the shell side, such as a perforated plate. The top zone (upper stage) of the reactor is designed to operate as an isothermal reactor with cooling by evaporation of a boiling coolant. The bottom zone (lower stage) is designed to be cooled by a coolant at below its boiling temperature.

A further aspect of the present invention provides a method for operating the two-stage reactor to achieve high rate of reaction in the higher temperature upper isothermal zone and high conversion of the reactant in the lower temperature lower zone. The combination of high rate and high conversion is achieved in a much smaller reactor and smaller amount of catalyst than can be achieved in isothermal reactors. Reactants are fed to the tubes in the reactor from the top and flow downward as the reaction proceeds. Coolant flows up from its feed point in the reactor. The top zone of the reactor is operated semi-isothermally, while liquid boils in the shell side at constant temperature. The top zone is operated at elevated temperature to achieve high rate of reaction, while it approaches equilibrium. The bottom zone operates as a counter-current cooling zone where the process gas in the tubes is cooled by counter flow of colder liquid coolant on the shell-side. The lower temperature of the process in the bottom zone results in improved equilibrium (favoring the forward reaction) and better conversion of the reactants to products. The high efficiency of the reactor of the current invention is a result of a design that incorporates two reaction zones in one reactor vessel allowing for both high reaction rate (top zone) and equilibrium conditions that favor the forward reaction (bottom zone).

Other aspects of the invention, including apparatuses, devices, processes, and the like which constitute part of the invention, will become more apparent upon reading the following detailed description.

### BRIEF DESCRIPTION OF THE DRAWINGS

The accompanying drawings are incorporated in and constitute a part of the specification. The drawings, together with the general description given above and the detailed description, serve to explain the principles of the invention. In such drawings:

FIG. 1 shows a schematic of the reactor of the present invention and associated systems;

FIG. 2 shows a cross-section of the reactor; and

FIG. 3 shows a typical temperature (T) profile of the reactor.

### DETAILED DESCRIPTION

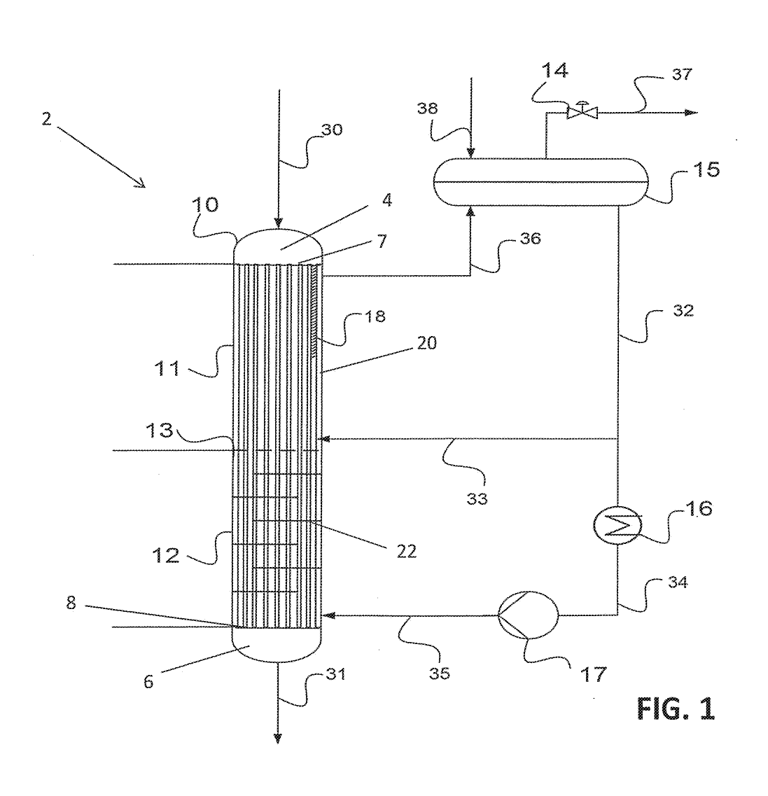

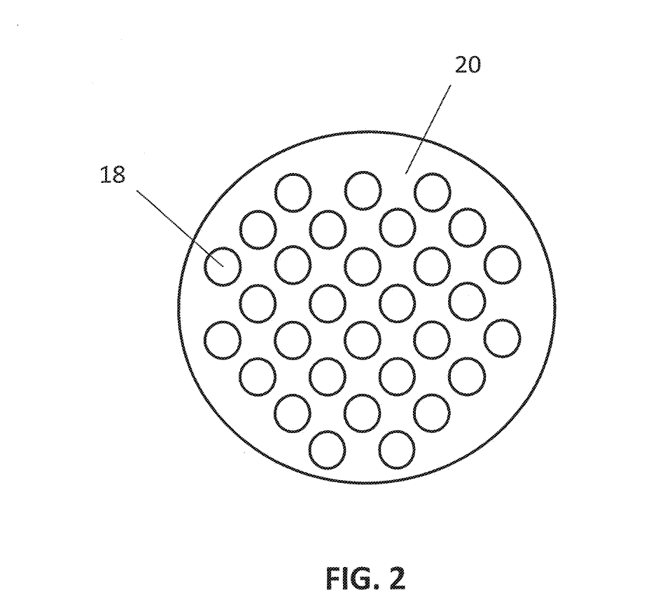

Referring to FIGS. 1-2, the reactor of the present invention is a shell and tube reactor 2, which generally contains a vessel 10 containing a plurality of tubes 18 extending axially inside the vessel 10. The tubes 18 extend a majority of the length of the vessel 10, and are designed to carry out an exothermic and reversible reaction in its lumen, while the shell side 20 of the reactor 2 is designed to allow a coolant to flow therethrough. A perforated plate 13 divides the reactor in to a top zone 11 and a bottom zone 12. The perforated plate 13 is used as a distributer of the flow from the lower to the upper zone and is designed to reduce or to minimize reverse flow in the shell side 20, from the upper to the lower zone. The perforated plate 13 impedes the flow of liquid on the shell side 20 of the reactor, but does not impede flow in the tubes 18. The reactor 2 also contains a top head 4 for feeding reactant(s) into the tubes 18 and a bottom head 6 for collecting reaction product(s) and unreacted reactant(s) from the tubes 18. A top tube sheet 7 separates the top head 4 from the shell side 20 of the reactor, such that fluid communication between the top head 4 and the lumen of the tubes 18 is preserved, but fluid communication between top head 4 and the shell side 20 is obstructed. Thus, reactants entering the top head 4 flows into the tubes 18, but not the shell side 20. Likewise, the coolant on the shell side 20 is also prevented from entering the top head 4 by the top tube sheet 7. A bottom tube sheet 8 also similarly separates the bottom head 6 from the shell side 20. Here, the reactant mixture inside the tubes can enter the bottom head 6, but coolant from the shell side 20 cannot.

The tubes 18 may contain catalysts therein to catalyze the exothermic, reversible reaction. In an exemplary embodiment, the reaction is the synthesis of methanol (product) from synthesis gas or syngas (reactants). Syngas typically contains CO, CO₂ and H₂ as the active species and CH₄ and N₂ as inert species. The syngas typically contains excess H₂ to achieve high conversion of the COx species (CO and CO₂). The syngas-to-methanol reactions are all exothermic and reversible as shown for reactions 1, 2 and 3 below.

CO+3H₂<----<CH₃OH+Heat 1)

CO₂+3H₂<---->CH₃OH+H₂O+Heat 2)

CO+H₂O<---->CO₂+H₂+Heat 3)

To enhance the syngas-to-methanol reactions, a catalyst may be used, e.g., a metal catalyst, typically copper/zinc based catalyst.

In operation, the top zone 11 of the reactor 2 is a semi-isothermal zone with reactant(s) (gas or liquid) stream 30 entering at the top and flows downward in the tubes 18. The reaction inside the tubes 18 is exothermic; and the heat generated is transferred to the shell side 20 where cooling boiling liquid, such as water, flows upwards. The makeup coolant 38 may enter the system into drum 15 and through natural circulation, caused by heating and boiling in the shell side 20 in the top zone 11, flows through pipes 32 and 33 into the shell side 20 just above the perforated plate 13. The cooling in the top zone 11 is accomplished mainly by boiling so that the temperature of the boiling coolant in the shell side 20 is approximately constant (hence isothermal) and may be controlled by the back pressure control valve 14. As a result of the high rate of the natural circulation, the entire loop comprising of the coolant in the drum 15, liquid coolant in streams 32, 33 and liquid and vapor coolant in stream 36 and 37 are approximately at the same temperature (within 20° C., preferably 10° C., more preferably 5° C.). For the syngas-to-methanol reactions, the top zone should be run at an average typical temperature of about 230 to about 270° C., preferably about 240 to about 260° C.

The bottom zone 12 of the reactor 2 is a counter-flow zone with the reaction mixture (gas or liquid) in the tubes 18 from the upper zone flowing downward and exiting the reactor 2 at the bottom in stream 31. The reaction inside the tubes 18 is exothermic; and the heat generated is transferred to the shell side where the coolant (e.g. water) flows upwards counter-currently to the gas flow in the tubes 18. The coolant in the bottom zone 12 is fed to the shell side 20 of the bottom zone 12 from stream 35, which is a fraction of stream 32 flowing by the action of the pump 17 and cooled in the heat exchanger 16. The heat in the heat exchanger 16 may be removed by cooling water or air, or recovered and used in other parts of the process. The desired temperature in the bottom zone 12 depends on the reaction under consideration. In an exemplary embodiment, for the syngas-to-methanol reactions, the desired temperature at the tubes outlet is preferably about 190 to about 230° C. To achieve that desired temperature, the coolant temperature of stream 35, when it enters the shell side 20, is preferably about 2 to about 10° C. lower than the desired temperature of the exit stream of the tubes 18.

Stream 35 is cooled sufficiently below the boiling temperature of the coolant at the operating pressure of the system, so that in the shell side 20 of the bottom zone 12, there is no boiling and heat is transferred from the tubes 18 to the coolant by a conduction/convection in a counter flow heat exchanging mechanism. In certain embodiments, baffles 22, as best as shown in FIG. 1, may be installed in the bottom zone 12 to enhance heat transferred.

The perforated plate 13 separating the top zone 11 and the bottom zone 12 is designed to introduce flow resistance to the up flowing liquid coolant, so that it enters the upper boiling zone 11 in a reasonable uniform flow pattern. In addition, the perforated plate prevents back flow of boiling liquid from the top zone 11 to the bottom zone 12.

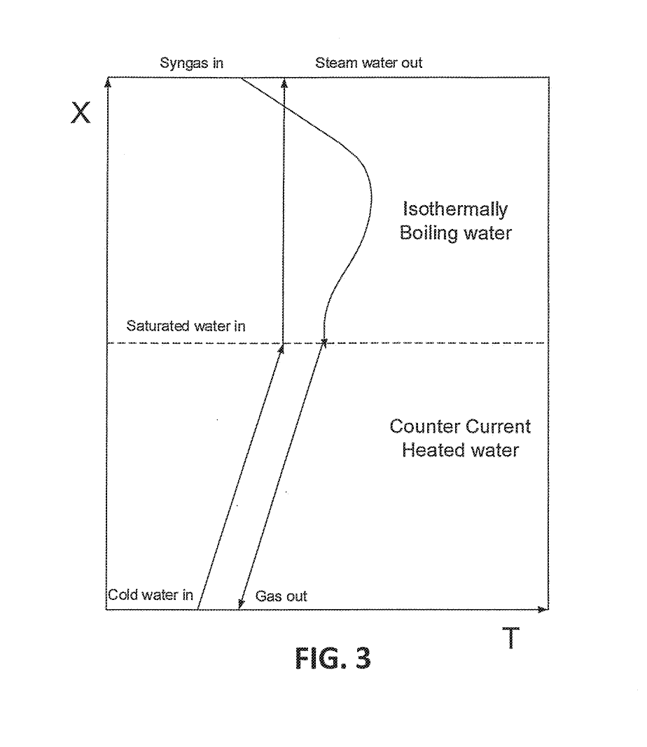

FIG. 3 shows a typical temperature profile in the reactor of the present invention. In that figure, X (y-axis) indicates the height of the reactor and T (x-axis) indicates the temperature. In the upper zone, as illustrated in FIG. 3, the coolant is boiling at constant temperature in the shell side 20, while the reactant(s) entering the tubes at the top is typically cooler than the boiling water. Due to the heat generated by the reaction, the temperature inside the tubes 18 quickly increases and reaches a maximum. The maximum temperature in the tubes occurs where the reaction rate and the heat generation are the highest. As the reaction progresses, the rate of heat generation decreases, and the difference between the gas temperature in the tubes 18 and the boiling temperature of the coolant in the shell side 20 decreases.

In the bottom zone 12 of the reactor 2, the sub-saturated coolant enters the shell side 20 at the bottom (via stream 35) and flows upward while absorbing heat from the tubes. The coolant exiting the bottom zone 12 (and going through the perforation plate 13 to the top zone 11) is warmer than when it enters the bottom zone 12, and preferably, but not necessarily, at or close to the saturation temperature, as in the top zone 11. The rate of reaction in the tubes 18 in the bottom zone 12 is lower than in the top zone 11 due to the fact that a large portion of the reactants have already been consumed. Cooling the reaction mixture in the bottom zone 12 results in favorable equilibrium conditions (equilibrium favoring the product(s)) and additional conversion of the reactant(s) to product(s).

The foregoing detailed description of the certain exemplary embodiments has been provided for the purpose of explaining the principles of the invention and its practical application, thereby enabling others skilled in the art to understand the invention for various embodiments and with various modifications as are suited to the particular use contemplated. This description is not necessarily intended to be exhaustive or to limit the invention to the precise embodiments disclosed. The specification describes specific examples to accomplish a more general goal that may be accomplished in another way. Accordingly, it is intended that the invention be limited only to the extent required by the appended claims and the applicable rules of law.

What is claimed is:

1. A chemical reactor comprising a vessel having
a. a plurality of tubes extending axially inside the vessel defining a tube side and a shell side;
b. a perforated plate dividing the shell side into a top zone which is configured to operate at a first temperature range, and a bottom zone which is configured to operate a second temperature range lower than the first temperature range.

2. The chemical reactor of claim 1, wherein the shell side is fluidly connected to a drum containing a coolant.

3. The chemical reactor of claim 2, wherein the coolant is water.

4. The chemical reactor of claim 1, wherein an exothermic and reversible reaction takes place inside the tubes.

5. The chemical reactor of claim 4, wherein the exothermic and reversible reaction produces methanol from syngas.

6. The chemical reactor of claim 1, wherein chemicals inside the tubes flows from the top zone to the bottom zone.

7. The chemical reactor of claim 1, wherein a coolant in the shell side flows counter current to the chemicals inside the tubes.

8. The chemical reactor of claim 1, wherein the top zone is cooled by a coolant evaporating on the shell side.

9. The chemical reactor of claim 1, wherein the tubes are filled with a catalyst.

10. The chemical reactor of claim 1, wherein the bottom zone is cooled by a liquid coolant below its boiling point.

11. A process for making a product from an exothermic and reversible reaction, comprising the steps of
a. providing the chemical reactor of claim 1;
b. conducting the exothermic and reversible reaction in the tubes while flowing reactant from top to bottom;
c. cooling the top zone by boiling a liquid coolant; and
d. cooling the bottom zone by the liquid coolant below its boiling point.

12. The process of claim 11, wherein the flow of the liquid coolant is counter current to the flow inside the tube.

13. The process of claim 11, wherein the liquid coolant is water.

14. The process of claim 11, wherein the top zone of the shell side operates at a constant temperature along a length of the reactor.

15. The process of claim 11, wherein the liquid coolant is fed to the top zone at a point above and adjacent to the perforated plate.

16. The process of claim 11, wherein the liquid coolant fed to the top zone is a saturated liquid.

17. The process of claim 11, wherein the tubes are filled with a catalyst.

18. The process of claim 11, wherein the exothermic and reversible reaction produces methanol from syngas.

19. The process of claim 11, wherein the liquid coolant is fed to the bottom zone at a point above and adjacent to a bottom head.

20. The process of claim 11, wherein the coolant is stored in a drum, is and fed to the upper zone as a saturated liquid, and is cooled in a heat exchanger prior to being fed to the bottom zone.
